(19) United States
(12) Patent Application Publication
(10) Pub. No.: US 2004/0019070 A1
(43) Pub. Date: Jan. 29, 2004

(54) Pyrrole-condensed morphinoid derivatives

(76) Inventors: Giulio Dondio, Milan (IT); Stefania Gagliardi, Milan (IT); Davide Graziani, Milan (IT)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/398,354

(22) PCT Filed: Oct. 5, 2001
(86) PCT No.: PCT/EP01/11556

(30) Foreign Application Priority Data
Oct. 12, 2000 (GB) 0025057.1

(51) Int. Cl. A61K 31/485; C07D 489/12

(52) U.S. Cl. 514/278; 546/16; 546/39; 514/279

(57) ABSTRACT

A compound or a salt or solvate thereof, of formula (I):

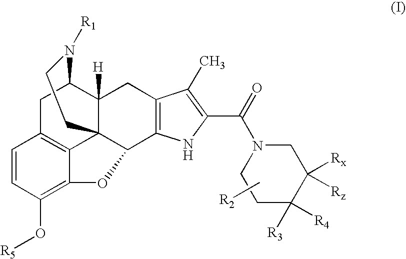

in which:

R₁ is hydrogen, alkenyl or alkyl;

R₂ is hydrogen or one or more alkyl groups;

R₅ is hydrogen or alkyl;

Rₓ is hydrogen or alkyl; or Rₓ together with R₃ represent a bond;

when Rₓ is hydrogen or alkyl, then R_{z} is hydrogen or alkyl; and

R₃ is selected from hydrogen, optionally substituted alkyl, aryl, aryl-alkyl, cycloalkyl and heterocyclyl; or from hydrogen or an optionally substituted alkyl, aryl, aryl-alkyl, cycloalkyl or heterocyclic group linked to the piperidine ring via>C═O, —O—CO—, —O—CO—NH—, —CO—NH—, —NH—CO— or >SO₂; and

R₄ is selected from hydrogen, alkyl, —CN, —COR₁ and —OR₁ (where R₁ is as defined above) or a —NR₆R₇ group, where R₆ and R₇ are selected independently from hydrogen and alkyl, or R₆ is —COR₈ where R₈ is hydrogen, alkyl or optionally substituted aryl, aryl-alkyl, cycloalkyl or heterocyclyl;

or R₃ and R₄ together form a spiro-cyclylalkyl, spiro-heterocyclyl or an unsaturated spiro-carbocyclyl group;

when Rₓ together with R₃ represent a bond then:

R₄ is a group R₄ₓ and R_{z} is a group R₃ₓ;

wherein R₃ₓ and R₄ₓ are independently selected from hydrogen or optionally substituted alkyl, aryl, aryl-alkyl, cycloalkyl or heterocyclyl;

The present invention is concerned with novel morphinoid compounds, processes for their preparation and their use in medicine.

The presence of at least three populations of opioid receptors (mu, delta and kappa) is now well established and documented and all three appear to be present in the central and peripheral nervous system of many species including man (J. A. H. Lord et al., Nature 1977, 267, 495).

Activation of all three opioid receptor subtypes can lead to antinociception in animal models. In particular, studies with peptidic delta agonists have indicated that activation of the delta receptor produces antinociception in rodents, primates and can induce clinical analgesia in man (D. E. Moulin et al, Pain, 1985, 23, 213). Evidence exists that suggest a lesser propensity of delta agonists to cause the usual side-effects associated with mu and kappa activation (Galligan et al, J. Pharm. Exp. Ther., 1984, 229, 641).

WO 96/02545 and WO 97/25331 (Smith ine Beecham SpA) disclose substituted monoheterocycle-condensed morphinoid derivatives which are potent and selective delta opioid agonists and antagonists, including (in WO 97/25331) the compound:

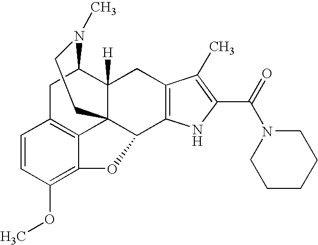

We have discovered that certain novel morphinoid compounds are surprisingly more potent and selective than known delta opioid receptor ligands and therefore of potential therapeutic utility as analgesics and antihyperalgesics for different pain conditions; immunosuppressants to prevent rejection following organ transplants and skin grafts; anti-allergic and anti-inflammatory agents; brain cell protectants; agents for treating drug and alcohol abuse, cardiovascular and respiratory diseases, cough, mental illness and epilepsy; agents for treating gastrointestinal disorders such as gastritis, diarrhoea, and irritable bowel syndrome; and, in general, for the treatment of those pathological conditions which customarily can be treated with agonists and antagonists of the delta opioid receptor.

According to the present invention there is provided a compound, or a solvate or salt thereof, of formula (I):

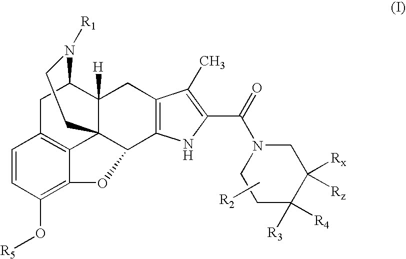

in which:

R₁ is hydrogen, alkenyl or alkyl;

R₂ is hydrogen or one or more alkyl groups;

R₅ is hydrogen or alkyl;

Rₓ is hydrogen or alkyl; or Rₓ together with R₃ represent a bond;

when Rₓ is hydrogen or alkyl, then R_{z} is hydrogen or alkyl; and

R₃ is selected from hydrogen, optionally substituted alkyl, aryl, aryl-alkyl, cycloalkyl and heterocyclyl; or from hydrogen or an optionally substituted alkyl, aryl, aryl-alkyl, cycloalkyl or heterocyclic group linked to the piperidine ring via >C═O, —O—CO—, —O—CO—NH—, —CO—NH—, —NH—CO— or >SO₂; and

R₄ is selected from hydrogen, alkyl, —CN, —COR₁ and —OR₁ (where R₁ is as defined above) or a —NR₆R₇ group, where R₆ and R₇ are selected independently from hydrogen and alkyl, or R₆ is —COR₈ where R₈ is hydrogen, alkyl or optionally substituted aryl, aryl-alkyl, cycloalkyl or heterocyclyl;

or R₃ and R₄ together form a spiro-cyclylalkyl, spiro-heterocyclyl or an unsaturated spiro-carbocyclyl group;

when Rₓ together with R₃ represent a bond then:

R₄ is a group R₄ₓ and R_{z} is a group R₃ₓ;

wherein R₃ₓ and R₄ₓ are independently selected from hydrogen or optionally substituted alkyl, aryl, aryl-alkyl, cycloalkyl or heterocyclyl;

or R₃ₓ and R₄ₓ together form an aliphatic or aromatic group, or a heterocyclyl group containing one or two heteroatoms selected from N, S and O;

provided that when R₅ is methyl and R₃ and R₄ are both hydrogen, then R₂ is not hydrogen.

When Rₓ is hydrogen or alkyl and R_{z} is hydrogen or alkyl, then the moiety:

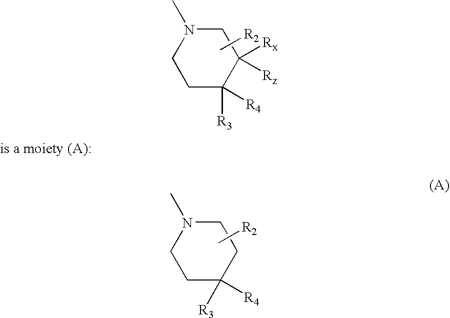

wherein R₂, R₃ and R₄ are as hereinbefore defined.

When Rₓ together with R₃ represents a bond then the moiety:

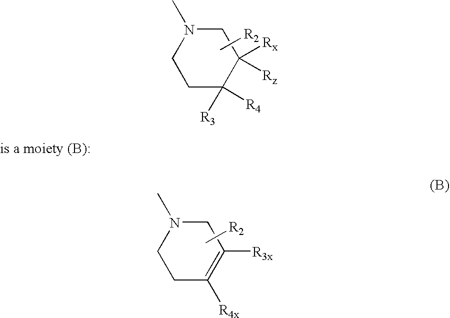

wherein R₂, R₃ₓ and R₄ₓ are as hereinbefore defined.

In an alternative aspect according to the present invention, there is provided a compound, or a solvate or salt thereof, of formula (Ia):

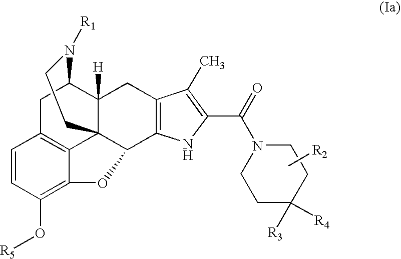

in which:

R₁ is hydrogen or alkyl;

R₂ is hydrogen or one or more alkyl groups;

R₃ is selected from hydrogen, optionally substituted alkyl, aryl, aryl-alkyl, cycloalkyl and heterocyclyl; or from hydrogen or an optionally substituted alkyl, aryl, aryl-alkyl, cycloalkyl or heterocyclic group linked to the piperidine ring via >C═O, —O—CO—, —O—CO—NH—, —CO—NH—, —NH—CO— or >SO₂;

R₄ is selected from hydrogen, alkyl, —CN, and —OR₁ group (where R₁ is as defined above) or a —NR₆R₇ group, where R₆ and R₇ are selected independently from hydrogen and alkyl, or R₆ is —COR₈ where R₈ is hydrogen, alkyl or optionally substituted aryl, aryl-alkyl, cycloalkyl or heterocyclyl;

or R₃ and R₄ together form a spiro-cycloalkyl or spiro-heterocyclyl group; and

R₅ is hydrogen or alkyl;

provided that when R₅ is methyl and R₃ and R₄ are both hydrogen, then R₂ is not hydrogen.

In a particular aspect the compound of formula (I) has a piperidine moiety (A):

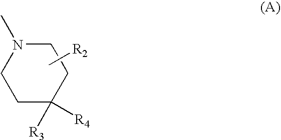

A particular set of compounds are those having a moiety (A) wherein when R₃ and R₄ are both hydrogen then R₂ is not hydrogen.

R₁ is preferably hydrogen, methyl, ethyl or allyl.

R₅ is suitably hydrogen or methyl. In a further aspect R₅ can be hydrogen. Suitably R₅ can be methyl, alternatively R₅ can be C₂₋₆alkyl.

R₁ and R₅ are typically hydrogen or alkyl, especially methyl.

Typical R₂ groups include hydrogen and alkyl, especially methyl, which may be present as dimethyl, gem-dimethyl or bis(gem-dimethyl). Two R₂ groups may link to form an alkylene bridge, suitably a C₂₋₄alkylene bridge, more suitably the bridge is ethylene.

R₃ groups are typically hydrogen, or optionally substituted aryl, aryl, aryl-alkyl, cycloalkyl or heterocyclyl linked to the piperidine ring directly or via >C═O, —O—CO—, —O—CO—NH—, —CO—NH—, —NH—CO—, or >SO₂, especially via >C═O, —O—CO—NH— and —CO—NH—.

When the R₃ substituent is one of the above groups connected to the piperidine ring by a linking group, suitable substituents include ethyl-OOC—, t-butyl-O—CO—NH—, methyl-CO—NH—, t-butyl-O—CO—, i-butyl-CO—, n-butyl-CO—, i-pentyl-CO—, phenyl-CO—, benzyl-CO—, phenylethyl-CO—, cyclohexyl-CO—, methyl-NH—CO, ethyl-NH—CO—, methyl-SO₂— and formyl. Especially suitable are ethyl-OOC—, t-butyl-O—CO—NH—, methyl-CO—NH—, and phenyl-CO,more especially ethyl-OOC—, t-butyl-O—CO—NH—, and phenyl-CO.

Suitable optional substituents for cyclic R₃ groups include oxo, dioxymethylene, bromo, chloro, fluoro, hydroxy, cyano, methyl, methoxy, t-butyl and phenyl, especially oxo, bromo, chloro, fluoro, methoxy, and phenyl.

R₄ is suitably hydrogen, —CN, OH, —COR₈, especially where R₈ is alkyl, for example methyl, or a —NR₆R₇ group, especially where R₆ is —COR₈, such as —NHCOalkyl, especially —NHCOMe.

R₄ is typically hydrogen, but may also advantageously be a —NR₆R₇ group, especially where R₆ is —COR₈, such as —NHCOalkyl, especially —NHCOMe.

The R₃ and R₄ groups may also be linked together to form a spirocyclic group, especially spiro-heterocyclyl, such as spiro-imidazolinyl and spiro-indenyl, especially spiro-imidazolinyl.

Typically R₃ₓ and R₄ₓ are selected from hydrogen and optionally substituted aryl, e.g. phenyl.

When R₃ₓ and R₄ₓ combine to form a group, the group is typically a heterocyclyl group.

When R₃ₓ and R₄ₓ combine to form a heterocyclyl group, a typical group is indolyl.

When X is a tetrahydropyridyl moiety, R₂ is preferably hydrogen.

A particular compound is [8R-(4bS*,8α, 8a β,12b β)]-1-(4-phenylpiperidin-1-yl)carbonyl-1-methoxy-7,10-dimethyl-5,6,7,8,12,12b-hexahydro-(9H)4,8-methanobenzofuro[3,2-e]-pyrrolo[2,3-g]isoquinoline or a salt or solvate thereof.

The compounds of formula (I) or their salts or solvates, are preferably in pharmaceutically acceptable or substantially pure form. By pharmaceutically acceptable form is meant, inter alia, of a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in a pharmaceutical composition. In the case of salts and solvates the additional ionic and solvent moieties must also be non-toxic.

Examples of pharmaceutically acceptable salts of a compound of formula (I) include the acid addition salts with the conventional pharmaceutical acids, for example, maleic, hydrochloric, hydrobromic, phosphoric, acetic, fumaric, salicylic, citric, lactic, mandelic, artaric, succinic, benzoic, ascorbic and methanesulphonic.

The compounds of formula (I) may exists in more than one stereoisomeric form, and the invention extends to all such forms as well as to their mixtures thereof, including racemates.

Alkyl groups, including alkyl groups that are part of, for example, alkoxy or acyl groups, typically contain 1 to 6 carbon atoms, and may be linear or branched, such as methyl, ethyl, i-propyl, t-butyl or i-pentyl, and optionally substituted by hydroxyl. Alkenyl groups typically contain 2 to 6 carbon atoms, and may be linear or branched, such as allyl. Aryl groups are typically phenyl, but may include bicyclic groups such as naphthyl. Cycloalkyl groups typically contain from 3 to 7 carbon atoms, for example cyclohexyl. Unsaturated carbocyclic groups typically contain from 4 to 7 carbon atoms, but may include bicyclic groups such as indenyl. Heterocyclic groups may be monocyclic 5 to 7 membered rings containing up to three heteroatoms, such as pyridyl or imidazolyl, or fused with another aliphatic, aromatic or heterocyclyl ring containing one or two heteroatoms selected from N, S and O to give a bicyclic system, especially heterocyclic rings fused to benzene rings, such as indolyl, benzoxazolyl or benzimidazolyl. Aryl, cycloalkyl and heterocyclic groups may be optionally substituted by up to three substituents, which may suitably be selected from aryl, alkyl, alkoxy, halogen, hydroxy, oxo and cyano, or by linked substituents such as dioxymethylene.

The compounds of formula (I), or salts or solvates thereof, may be prepared by the methods illustrated in the following general reaction schemes, or by modification thereof, using readily available starting materials, reagents and conventional synthetic procedures. If a particular enantiomer of a compound of the present invention is desired, it may be synthesised starting from the desired enantiomer of the starting material and performing reactions not involving racemization processes or it may be prepared by chiral synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxy, diastereomeric salts are formed with an appropriate optically active acid or base, followed by resolution of diastereomeric salts by fractional crystallisation and subsequent recovery of the pure enantiomers.

Salts or solvates of the compounds of formula (I) which are not pharmaceutically acceptable may be useful as intermediates in the production of pharmaceutically acceptable salts or solvates. Accordingly such salts or solvates also form part of this invention.

The compounds of formula (I) and pharmaceutically acceptable derivatives thereof may be prepared by the processes described hereinafter, said processes constituting a further aspect of the invention. In the following description, the groups R₁, R₂, R₃, R₄, Rₓ, R₃ₓ, R₄ₓ, R_{z}, and R₅ are as defined for compounds of formula (I) unless otherwise stated.

Compounds of formula (I) may be prepared by the reaction of acyl compounds of formula (II) with compounds of formula (III) using the following reaction scheme 1:

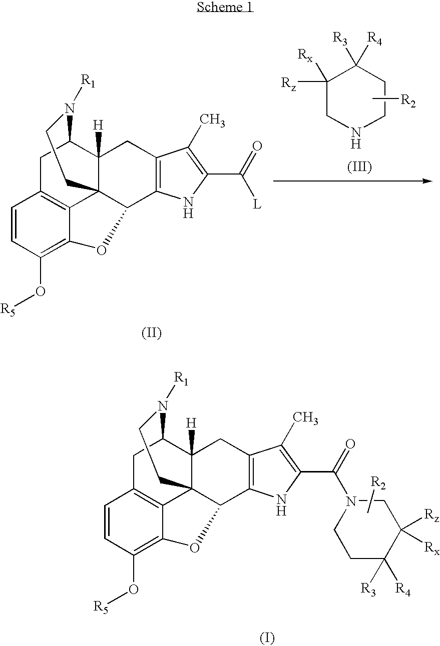

wherein L is a leaving group, for example halo, especially chloro;

followed, if so desired, by conversion to a salt and/or solvate thereof.

The reaction of a compound of formula (II) with a compound of formula (III) is suitably carried out in the presence of a base, for example an organic base, such as an amine, for example triethylamine, diisopropylethylamine, pyridine or dicyclohexylamine. A particular amine that may be mentioned is triethylamine. The reaction is suitably carried out in the presence of a solvent, for example an aprotic solvent such as N,N-dimethylformamide, tetrahydrofuran, N-methylpyrrolidinone, or dimethoxyethane; or a chlorinated solvent such as dichloroethane or dichloromethane. A particular solvent that may be mentioned is tetrahydrofuran. Suitable reaction temperatures include 10-40° C., more particularly 15-25° C.

Compounds of formula (II) may be prepared according to processes known in the art for the preparation of acyl groups, for example S. Patai, The Chemistry of Acyl Halides, pp 35-78, Interscience, New York, 1972. The compounds of formula (II) when L is chloro may be obtained for example by reacting a ketone of formula (IV) with a hydrazone of formula (V), in the presence of Zn and CH₃COONa in CH₃COOH as solvent (Khimiya Geterot. Soed., 1972, 342), followed by treatment with sodium hydroxide to give the sodium carboxylate. Conversion to the corresponding acyl chloride is achieved by treatment with oxalyl chloride, as shown in scheme 2:

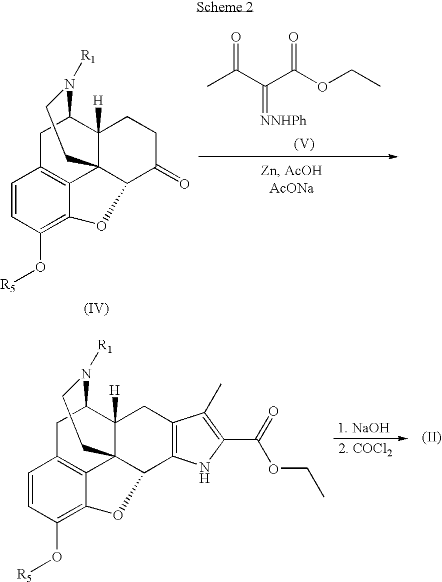

Suitable starting materials of formula (IV) include the morphinan hydrocodone (R₁ and R₅ are methyl), which is commercially available, and other compounds which are known in the literature. The other substituents specified for R₁ and R₅ in formula (I) may be incorporated and/or converted by conventional substitution reactions before or after the coupling of compounds (II) with (III). For example, a compound of formula (I) or formula (II) in which R₁ is alkyl or alkenyl may be converted into another compound of formula (I) or formula (II) respectively in which R₁ is hydrogen by conventional methods. Similarly a compound of formula (I) or formula (II) in which R₁ is hydrogen may be converted into a compound of formula (I) or formula (II) in which R₁ is alkyl or alkenyl by conventional substitution reactions. Similarly compounds of formula (I) or formula (II) in which R₅ is alkyl may be converted to the corresponding compounds in which R₅ is hydrogen, and compounds in which R₅ is hydrogen may be converted to corresponding compounds in which R₅ is alkyl.

The compounds of formula (I) may be alternatively obtained starting from ketones of formula (IV) and hydrazones of formula (VI) prepared using as starting material compounds prepared by reacting ethylacetoacetate with the appropriate substituted piperidines or tetrahydropyridines of general formula (III). The resulting acetoacetamides of general formula (VII) are in turn reacted with phenyldiazonium salt to obtain the corresponding hydrazones of general formula (VI) as shown in scheme 3:

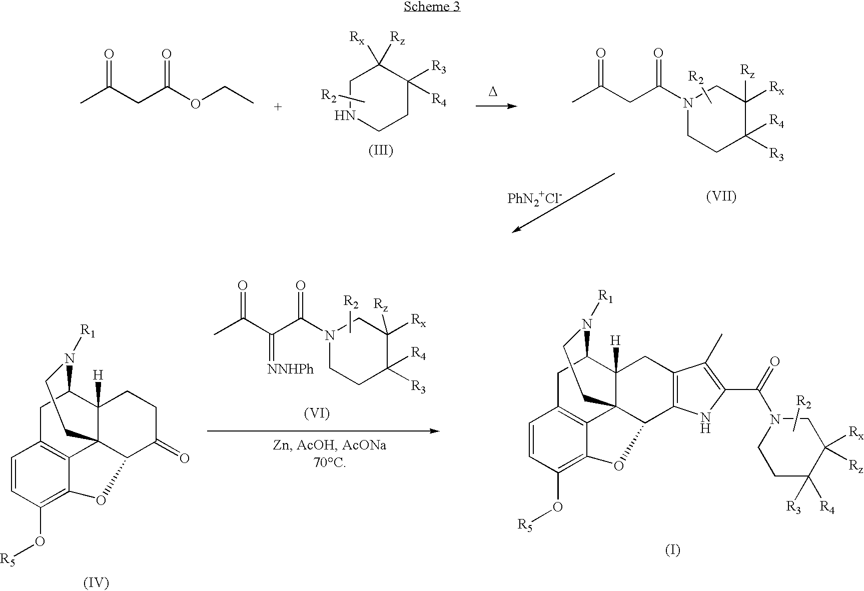

The substituted piperidines and tetrahydropyridines of formula (III) are either commercially available or readily synthesised by conventional methods from commercially available materials.

In a further aspect the present invention provides for novel intermediates of formulae (II), (II), (IV), (V), (VI) and (VII).

The compounds of formula (I) may be converted into their pharmaceutically acceptable salts by reaction with the appropriate reagent, for example organic or mineral acids.

Solvates, including hydrates, of the compounds of formula (I) may be formed by crystallisation or recrystallisation from the appropriate solvent For example, hydrates may be formed by crystallisation or recrystallisation from aqueous solutions, or solutions in organic solvents containing water.

In general compounds of formula (I) acting as selective delta receptor ligands may be useful as analgesics and antihyperalgesics for different pain conditions, immunosuppressants to prevent rejection in organ transplant and skin graft, anti-allergic and anti-inflammatory agents, brain cell protectant, for the treatment of drug and alcohol abuse, to decrease gastric secretion, for the treatment of diarrhoea, cardiovascular and respiratory diseases, cough and respiratory depression, mental illness, epileptic seizures and other neurologic disorders (herein after referred to as “Conditions”). In particular, the activity of the compounds of formula (I) as delta agonists in standard tests indicates that they are of potential therapeutic utility as analgesic agents for the amelioration or elimination of pain.

Accordingly the present invention provides a method for the treatment and/or prophylaxis of one or more of the Conditions in mammals, particularly humans, which comprises administering to the mammal in need of such treatment and/or prophylaxis an effective amount of a compound of formula (I) or a pharmaceutically acceptable salt or solvate thereof.

The present invention also provides a compound of formula (I), or a pharmaceutically acceptable salt or solvate thereof, for use as an active therapeutic substance.

In particular the present invention also provides a compound of formula (I) for use in the treatment and/or propylaxis of one or more of the Conditions.

The present invention further provides a pharmaceutical composition comprising a compound of formula (I), or a pharmaceutically acceptable salt or solvate thereof, and a pharmaceutically acceptable carrier.

The present invention also provides the use of a compound of formula (I), or a pharmaceutically acceptable salt or solvate thereof, in the manufacture of a medicament for the treatment and or prophylaxis of one or more of the Conditions.

Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the Conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the Conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may administer to himself in a single dosage. Advantageously, the composition is suitable for oral, rectal, topical, parenteral, intravenous or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatin containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The compounds of this invention may also be administered by a non-oral route. In accordance with routine pharmaceutical procedure, the compositions may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

The compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the compounds of the invention comprises transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery to the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 5 to 1000 mg, preferably 20 to 1000 mg, and more preferably will contain from 30 to 500 mg, in particular 50, 100, 150, 200, 250, 300, 350, 400, 450, or 500 mg. The composition may be administered once or more times a day for example 2, 3 or 4 times daily, and the total daily dose for a 70 kg adult will normally be in the range 10 to 3000 mg, preferably 100 to 3000 mg. Alternatively the unit dose will contain from 2 to 20 mg of active ingredient and be administered in multiples, if desired, to give the preceding daily dose.

No unacceptable toxicological effects are expected with compounds of the invention when administered in accordance with the invention.

The activity of the compounds of the present invention as selective delta ligands is determined in radioligand binding assays as described below.

Pharmacological Data

The activity of the compounds of the present invention as selective delta ligands is determined in radioligand binding assays using cloned human delta, mu and kappa opioid receptors stably expressed in cell lines as described below.

CHO cells were subjected to stable transfection with cDNA encoding the human delta and mu opioid receptors. Clones were grown in suspension culture in serum free media. Selection was performed by growth in the absence of nucleotides.

Human kappa opioid receptors were stably expressed in HEK cells. Cells were grown in adhesion in E-MEM supplemented with 10% FBS and 2 mM L-glutamine, G418 was included for selection.

Membranes were prepared as previously described (J. Med. Chem. 1997, 40, 3192 ). The binding of the preferential delta ligand [³H]-[D-Ala²,D-Leu⁵]-enkephalin (DADLE) was evaluated at its K_{D} concentration (0.7 nM). The binding of the mu ligand [³H]-DAMGO (Eur. J. Pharmacol., 1989, 166, 213) and of the kappa ligand [³H]-U69593 (Excerpta Medica, 1990, 211) were carried out at 0.5 nM. Non specific binding was determined in the presence of 10 uM of naloxone. Binding data were expressed as percentage of inhibition and fitted the following equation: f(x)=100·X/(IC₅₀+X) where X is the cold drug concentration value. The IC₅₀ values obtained were used to calculate the inhibitory constants (Kᵢ) accordingly to the Cheng and Prusoff relation (Biochem. Pharmacol., 1973, 22, 3099).

The most potent compounds described in the present invention showed affinities for the delta receptor ranging from 0.3 to 10 nM with delta selectivity ranging from 15 to 400 times in respect to the other opioid receptor types. For example, the compounds of Examples 1 and 6 show a Ki delta=0.3 nM (Ki mu/Ki delta=267) and Ki delta=1.4 (Ki mu/Ki delta=220), respectively.

The following Example illustrates the preparation of the compounds of general formula (I) of the present invention. The compounds of the remaining Examples 1 to 3 and 5 to 29 are obtained using the same general procedure as that described for Example 4 (below), starting from the corresponding acyl chloride of general formula (II) and the corresponding known substituted piperidine or tetrahydropyridine of general formula (III). Procedures A and B illustrate the preparation of intermediates of formula II and V.

The compounds obtained in the Examples 1 to 29 are summarised in Tables 1 to 3.

Procedure A—Preparation of the Sodium Salt of [8R-(4bS*,8α,8a β,12b β)]-7,10-dimethyl-1-methoxy-1-5,6,7,8,12,12b-hexahydro-(9H)-4,8-methanobenzofuro[3,2-e]-pyrrolo[2,3-g]isoquinoline-11-carboxylic Acid

To a solution of hydrocodeinone (25 g, MW 335.8, 74 mmol) in 400 ml of AcOH, AcONa (20 g, MW 82, 224 mmol) and 3-oxo-2-(phenyl-hydrazono)-butyric acid ethyl ester (V, see below) (34.6 g, MW 234.25, 147 mmol) were added. The mixture was heated to 50-60° C. and then Zn (16 g, MW 65.4, 244 mmol) was added portionwise, maintaining the temperature below 90° C. The mixture was then heated to 100° C. for 8 hours. The slurry was poured into a mixture containing 30% NH₄OH (200 ml) and 400 g of brine and then extracted with AcOEt (3×200 ml). The organic layer was dried over Na₂SO₄ and the solvent was removed under vacuum. The residue was purified by chromatography on silica gel eluting with AcOEt/MeOH/NH4OH 90/10/1 to afford the [8R-(4bS*,8α, 8a β,12b β)]-7,10-dimethyl-1-methoxy-11-ethyloxycarbonyl-5,6,7,8,12,12b-hexahydro-(9H)-4,8-methanobenzofuro[3,2-e]-pyrrolo[2,3-g]isoquinoline (12 g, PM 408.5, 30 mmol).

A mixture of this compound (5 g, 12 mmol) and 1M NaOH (30 ml) in 60 ml of EtOH 96% was stirred at 80° C. for 5 hours. The solvent was evaporated under vacuum and the residue was triturated in water (30 ml). After filtration, the solid was dried under vacuum at 50° C. for one night affording the title sodium carboxylate (4 g, MW 402.4, 10 mmol). Conversion to an acyl chloride of formula II (R₁ and R₅ are Me) is achieved by treatment with a suitable agent such as oxalyl chloride (see Example below).

Procedure B—Preparation of 3-oxo-2-(phenyl-hydrazono)-butyric Acid Ethyl Ester (Compound V)

In a flask containing 250 ml of 5 N HCl at the temperature of 0-5° C., 46.5 g of aniline (0.5 moles) were added under nitrogen flow. Then a solution of 36.5 g of NaNO₂ (0.53 moles) in 125 ml of water was added dropwise maintaining the temperature at 5-10° C. At the end of the addition the solution was adjusted to pH 4-5 with solid sodium acetate. The obtained solution was then transferred in a dropping funnel and added dropwise to a solution of ethylacetoacetate (65 g, 0.5 moles) in 375 ml of EtOH (95°), 100 ml of water and sodium acetate (0.73 moles) maintaining the temperature below 5-10° C. Then the reaction was warmed up to room temperature, and it was further stirred for two hours. 500 ml of water were added and after 30 minutes stirring, the suspension was filtered under vacuum. The orange-yellow solid was dried under vacuum at room temperature overnight to obtain 68 g of the title compound.

### EXAMPLE

Preparation of [8R-(4bS*,8α,8a β,12b β)]-11-(4-phenylpiperidin-1-yl)carbonyl-1-methoxy-7,10-dimethyl-5,6,7,8,12,12b-hexahydro-(9H)-4,8-methanobenzofuro[3,2-e]-pyrrolo[2,3-g]isoquinoline (Example 4)

Oxalyl chloride (1.1 ml, 12 mmol) was added dropwise at 0° C. to a suspension of sodium salt prepared as described in procedure A (above) (0.5 g, MW 402, 1.2 mmol) in THF (30 ml). The mixture was stirred 4 hours at room temperature (or to complete solution) and then was concentrated under vacuum.

The residue was dissolved in THF (10 ml) and the solution of phenylpiperidine (mg 317, MW 234.17, 1.3 mmol) in ThF (2 ml) and TEA (500 μl, 3.6 mmol) were added dropwise. The mixture was stirred at room temperature for 4 hours, then the solvent was removed in vacuo. The residue was dissolved in AcOEt (20 ml×2) and washed with 1N NaOH (5 ml). The organic layer was dried over Na₂SO₄ and evaporated under vacuum. The product was purified by chromatography on silica gel eluting with AcOEt/MeOH/NH₄OH 90/10/1 to afford 250 mg of the title compound.

TABLE 1

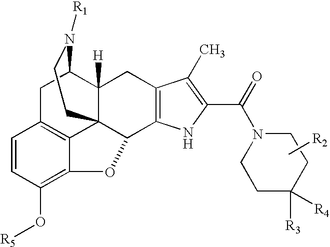

| | | | | | | | mass |
|---|---|---|---|---|---|---|---|
| | | | | | | | ESI POS; AQA; |
| | | | | | | | solvent: methanol/ |
| | | | | | | | spray 3 kV/ |
| | | | | | | [a]_{D}^{20§}/ | skimmer: 20 V/ |
| Ex | R₁ | R₂ | R₃ | R₄ | R₅ | (MP ° C.) | probe 135 C |
| 1 | Me | 2,2,6,6- | H | H | Me | (143-144) | * 503 (M+.); 488; |
| | | Me | | | | | 362;304; 126 |
| 2 | Me | 2,6- | H | H | Me | (177) | * 437 (M+.); 362; |
| | | (CH₂)₂ | | | | | 274 |
| 3 | Me | H | 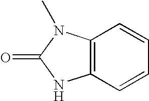 | H | Me | −370.9 (225-227) | * 579 (M+.), 364; 336; 218; 134; 82 |
| 4 | Me | H | Ph | H | Me | −400.6 | 523 (M+.) |
| | | | | | | (173.1-173.2) | |
| 5 | Me | H | NHCOO | H | Me | 414.8 | 563 (MH+) |
| | | | tBu | | | (210-219 | |
| 6 | Me | H | COPh | H | Me | −345.9 | 552 (MH+) |
| | | | | | | | (203.1-206.7) |
| 7 | Me | H | 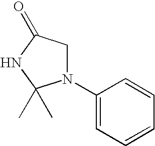 | | Me | — | 594 (MH+) |
| 8 | Me | H | 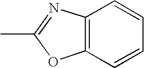 | H | Me | — | 565 (MH+) |
| 9 | Me | H | 4-Br—Ph | H | Me | — | 602 (MH+) |
| 10 | Me | H | 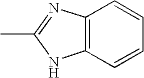 | H | Me | — | 564 (MH+) |
| 11 | Me | H | 2-Cl—Ph | H | Me | — | 558 (MH+) |
| 12 | Me | H | CH₂CH₂OH | H | Me | — | 492 (MH+) |
| 13 | Me | H | COOEt | H | Me | | 520 (MH+) |
| 14 | Me | H | Ph | NHCO | Me | — | 581 (MH+) |
| | | | | | Me | | |
| 15 | Me | H | 4-MeO—Ph | H | Me | | 554 (MH+) |
| 16 | Me | H | 3,5-F—Ph | H | Me | — | 560 (MH+) |
| 17 | H | H | Ph | H | Me | −289.1 | 510 (MH+) |
| 18 | Me | H | Ph | CN | Me | | 549 (MH+) |
| 19 | Me | H | Ph | CH₃CO | Me | | 566 (MH+) |
| 20 | Me | H | 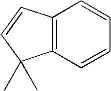 | | Me | | 548 (MH+) |
| 21 | Me | H | 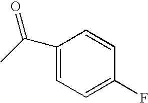 | H | Me | | 570 (MH+) |
| 22 | Me | H | Ph | NHMe | Me | | 553 (MH+) |
| 23 | Me | H | Ph | H | H | −482.35 | 509 (MH+) |
| 24 | Me | H | Cy | H | Me | | 530 (MH+) |
| 25 | Me | H | Ph | OH | Me | | 540 (MH+) |
| 26 | Et | H | Ph | H | Me | | 537 (MH+) |
| 27 | Allyl | H | Ph | H | Me | | 550 (MH+) |

^{§}c = 0.1%, solvent: MeOH;
*EI; TSQ 700; source 180 C; 70 V; 200 uA

Proton NMR data for Examples are as follows:

### Example 4

(CDCl₃) δ: 8.68 (s br, 1H); 7.30 (dd, 2H); 7.20 (m, 3H); 6.66 (d, 1H); 6.61 (d, 1H); 5.46 (s, 1H); 4.41−4.27(m, 2H); 3.80 (s, 3H); 3.22 (m, 1H); 3.05 (d, 1H); 2.99 (dq, 2H); 2.74 (tt, 1H); 2.58−2.26 (m, 5H); 2.44 (s, 3H); 2.03−1.59 (m, 7H); 1.95 (s, 3H).

### Example 6

(CDCl₃) δ: 8.63 (s br, 1H); 7.94 (d, 2H); 7.57 (dd, 1H); 7.47 (dd, 2H); 6.69 (d, 1H), 6.63 (d, 1H); 5.46 (s, 1H); 4.21 (m, 2H); 3.81 (s, 3H); 3.50 (m, 1H); 3.39 (m, 1H), 3.17−300 (m, 4H); 2.77−2.36 (m, 5H); 2.54 (s, 3H); 2.16−1.72 (m, 6H); 1.92 (s, 3H).

### Example 10

(CDCl₃-333 K) δ: 9.10 (s br, 1H); 8.65 (s br, 1H); 7.69 (s br, 1H); 7.38(s br, 1H); 7.21 (m, 2H); 6.67 (d, 1H); 6.60 (d, 1H); 5.43 (s, 1H); 4.38−4.20 (m, 2H); 3.80(s, 3H); 3.25−3.03 (m, 511); 2.59−2.31 (m, 4H); 2.46 (s, 3H); 2.16 (m, 2H); 2.06−1.80 (m,6H); 1.95(s, 3H).

### Example 17

(CDCl₃-333 K) δ: 8.59 (s br, 1H); 7.29 (dd, 2H); 7.20 (m, 3H); 6.68 (d, 1H) 6.61 (d, 1H); 5.41 (s, 1H); 4.40−4.28 (m, 2H); 3.81 (s, 3H); 3.49 (m, 1H); 3.13−2.69 (m, 7H); 2.44−2.33 (m, 2H); 1.95 (s, 3H); 1.95−1.61 (m, 8H).

### Example 26

(CDCl₃), δ: 8.55 (s br, 1H); 7.30 (dd, 2H); 7.21 (m, 3H); 6.66(d, 1H); 6.61 (d, 1H); 5.47 (s, 1H); 4.41−4.27 (m, 2H); 3.80 (s, 3H); 3.07−2.92 (m, 3H); 2.80−2.21 (m, 9H); 2.04−1.61 (m, 7H); 1.93 (s, 3H); 1.14 (t, 3H).

TABLE 2

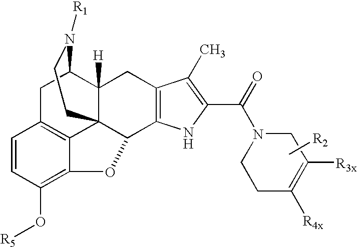

| | | | | | | | mass |
|---|---|---|---|---|---|---|---|
| | | | | | | | ESI POS; AQA; |
| | | | | | | | solvent: methanol/ |
| | | | | | | | spray 3 kV/ |
| | | | | | | [a]_{D}^{20§}/ | skimmer: 20 V/ |
| Ex. | R₁ | R₂ | R₃ₓ | R₄ₓ | R₅ | (MP ° C.) | probe 135 C |
| 28 | Me | H | 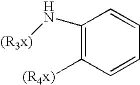 | | Me | | 535 (MH+) |
| 29 | Me | H | H | Ph | Me | | 522 (MH+) |

Table 3 illustrates intermediates of formula (II) and (III) for preparation of examples of the invention.

TABLE 3

| | | | |
|---|---|---|---|
| 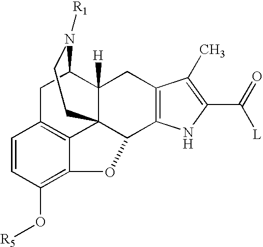 | (II) | 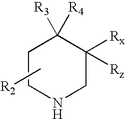 | (III) |

| | | | | Compound (III) | | |
|---|---|---|---|---|---|---|
| | Compound (II) | | | Rₓ,R_{y =} | | |
| Ex | L | R₁ | R₅ | R₂ | R₃ | R₄ |
| 1 | Cl | Me | Me | 2,2,6,6-Me | H | H |
| 2 | Cl | Me | Me | 2,6-(CH₂)₂ | H | H |
| 3 | Cl | Me | Me | H | 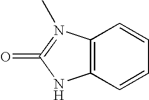 | H |
| 4 | Cl | Me | Me | H | Ph | H |
| 5 | Cl | Me | Me | H | NHCOOtBu | H |
| 6 | Cl | Me | Me | H | COPh | H |
| 7 | Cl | Me | Me | H | 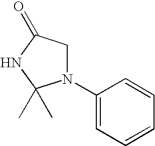 | |
| 8 | Cl Me Me | H | 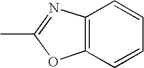 | H | | |
| 9 | Cl | Me | Me | H | 4-Br—Ph | H |
| 10 | Cl | Me | Me | H | 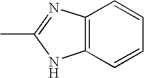 | H |
| 11 | Cl | Me | Me | H | 2-Cl—Ph | H |
| 12 | Cl | Me | Me | H | CH₂CH₂OH | H |
| 13 | Cl | Me | Me | H | COOEt | H |
| 14 | Cl | Me | Me | H | Ph | NHCOMe |
| 15 | Cl | Me | Me | H | 4-MeO—Ph | H |
| 16 | Cl | Me | Me | H | 3,5-F—Ph | H |
| 17 | Cl | H | Me | H | Ph | H |
| 18 | Cl | Me | Me | H | Ph | CN |
| 19 | Cl | Me | Me | H | Ph | CH₃CO |
| 20 | Cl | Me | Me | H | 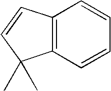 | |
| 21 | Cl | Me | Me | H | 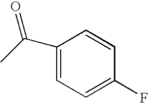 | H |
| 22 | Cl | Me | Me | H | Ph | NHMe |
| 23 | Cl | Me | H | H | Ph | H |
| 24 | Cl | Me | Me | H | Cy | H |
| 25 | Cl | Me | Me | H | Ph | OH |
| 26 | Cl | Et | Me | H | Ph | H |
| 27 | Cl | Allyl | Me | H | Ph | H |

| | | | | |
|---|---|---|---|---|
| 28 | Cl | Me | Me | R₂ is H; R₃ & Rₓ form a bond; R₄ is R₄ₓ and |
| | | | | R_{z} is R₃ₓ and together form the group: |
| | | | | 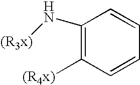 |
| 29 | Cl | Me | Me | R₂ is H; R₃ & Rₓ form a bond; R₄ is R₄ₓ and |
| | | | | is Ph; R_{z} is R₃ₓ and is H |

1. A compound or a salt or solvate thereof, of formula (I): 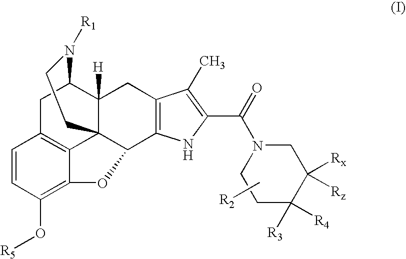
in which:
R₁ is hydrogen, alkenyl or alkyl;
R₂ is hydrogen or one or more alkyl groups;
R₅ is hydrogen or alkyl;
Rₓ is hydrogen or alkyl; or Rₓ together with R₃ represent a bond;
when Rₓ is hydrogen or alkyl, then R_{z} is hydrogen or alkyl; and
R₃ is selected from hydrogen, optionally substituted alkyl, aryl, aryl-alkyl, cycloalkyl and heterocyclyl; or from hydrogen or an optionally substituted alkyl, aryl, aryl-alkyl, cycloalkyl or heterocyclic group linked to the piperidine ring via >C═O, —O—CO—, —O—CO—NH—, —CO—NH—, —NH—CO— or >SO₂; and
R₄ is selected from hydrogen, alkyl, —CN, —COR₁ and —OR₁ (where R₁ is as defined above) or a —NR₆R₇ group, where R₆ and R₇ are selected independently from hydrogen and alkyl, or R₆ is —COR₈ where R₈ is hydrogen, alkyl or optionally substituted aryl, aryl-alkyl, cycloalkyl or heterocyclyl;
or R₃ and R₄ together form a spiro-cyclylalkyl, spiro-heterocyclyl or an unsaturated
spiro-carbocyclyl group;
when Rₓ together with R₃ represent a bond then:
R₄ is a group R₄ₓ and R_{z} is a group R₃ₓ;
wherein R₃ₓ and R₄ₓ are independently selected from hydrogen or optionally substituted alkyl, aryl, aryl-alkyl, cycloalkyl or heterocyclyl;
or R₃ₓ and R₄ₓ together form an aliphatic or aromatic group, or a heterocyclyl group
containing one or two heteroatoms selected from N, S and O;
provided that when R₅ is methyl and R₃ and R₄ are both hydrogen, then R₂ is not hydrogen.

2. A compound, or a solvate or salt thereof, of formula (Ia): 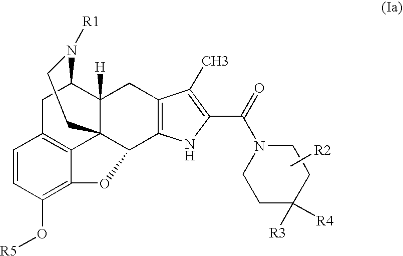
in which,
R₁ is hydrogen or alkyl;
R₂ is hydrogen or one or more alkyl groups;
R₃ is selected from hydrogen, optionally substituted alkyl, aryl, aryl-alkyl, cycloalkyl or heterocyclyl; or from hydrogen or an optionally substituted alkyl, aryl, aryl-alkyl, cycloalkyl or heterocyclic group linked to the piperidine ring via >C═O, —O—CO—, —O—CO—N—, —CO—N—, —N—CO— or >SO₂;
R₄ is selected from hydrogen, alkyl, —CN, an —OR₁ group (where R₁ is as defined above) or a —NR₆R₇ group, where R₆ and R₇ are selected independently from hydrogen and alkyl, or R₆ is —COR₈ where R₈ is hydrogen, alkyl or optionally substituted aryl, aryl-alkyl, cycloalkyl or heterocyclyl;
or R₃ and R₄ together form a spiro-cycloalkyl or spiro-heterocyclyl group; and
R₅ is hydrogen or alkyl;
provided that when R₅ is methyl and R₃ and R₄ are both hydrogen then R₂ is not hydrogen.

3. A compound according to claim 1 or claim 2 in which R₃ is selected from hydrogen, methyl, ethyl, i-propyl, i-butyl, t-butyl, n-butyl, i-pentyl, hydroxyethyl, phenyl, benzyl, phenylethyl, cyclohexyl, pyridyl, pyrimidyl, benzoxazolyl, benzimidazolyl, imidazolyl, ethyl-OOC—, t-butyl-O—CO—NH—, methyl-CO—NH—, t-butyl-O—CO—, i-butyl-CO—, n-butyl-CO—, i-pentyl-CO—, phenyl-CO—, benzyl-CO—, phenylethyl-CO—, cyclohexyl-CO—, methyl-NH—CO, ethyl-NH—CO—, methyl-SO₂— and formyl, where the cyclic R₃ groups are optionally substituted by oxo, dioxymethylene, bromo, chloro, fluoro, hydroxy, cyano, methyl, methoxy, t-butyl and phenyl, especially oxo, bromo, chloro, fluoro, methoxy, or phenyl.

4. A compound according to any one of claims 1 to 3 in which R₄ is selected from hydrogen, —CN, —OH, —COalkyl and —NHCOalkyl.

5. A compound according to claim 1 or claim 2 in which the R₃ and R₄ groups are linked together as spiro-imidazolinyl or spiro-indenyl.

6. A compound according to claim 1 in which R₃ₓ and R₄ₓ are selected from hydrogen and aryl.

7. A compound according to claim 1 in which R₃ₓ and R₄ₓ combine to form a heterocyclyl group.

8. A compound according to any one of claims 1 to 7 in which R₂ is selected from hydrogen, methyl, dimethyl, gem-dimethyl and bis(gem-dimethyl).

9. A compound according to any one of claims 1 to 7 in which two R₂ groups are linked to form an ethylene bridge.

10. A compound according to any one of claims 1 to 9 in which R₁ and R₅ are independently selected from hydrogen and methyl.

11. A compound according to claim 1 which is any one of compounds 1 to 29 in the Chemical Tables herein.

12. The compound [8R-(4bS*,8α, 8a β,12b β)]-11-(4-phenylpiperidin-1-yl)carbonyl-1-methoxy-7,10-dimethyl-5,6,7,8,12,12b-hexahydro-(9H)4,8-methanobenzofuro[3,2-e]-pyrrolo[2,3-g]isoquinoline or a salt or solvate thereof.

13. A method for the treatment and/or prophylaxis of one or more of the Conditions in mammals, particularly humans, which comprises administering to the mammal in need of such treatment and/or prophylaxis an effective amount of a compound according to any one of claims 1 to 12 or a pharmaceutically acceptable salt or solvate thereof.

14. A compound according to any one of claims 1 to 12, or a pharmaceutically acceptable salt or solvate thereof, as an active therapeutic substance.

15. Use of a compound according to any one of claims 1 to 12, or a pharmaceutically acceptable salt or solvate thereof, in the manufacture of a medicament for the treatment of one or more of the Conditions.

16. A process for the preparation of a compound of formula (I) according to claim 1 which process comprises:
reacting a compound of formula (II): 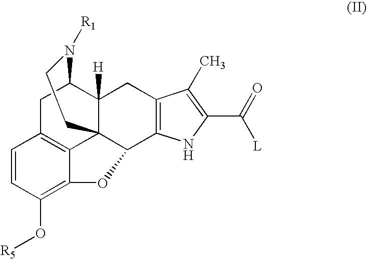
with a compound of formula (III): 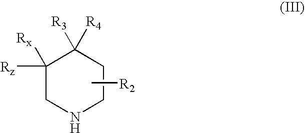
and, if desired, converting to a salt and/or solvate thereof;
wherein R₁, R₂, R₃, R₄, Rₓ, R_{z}, and R₅ are as defined in claim 1; and
L is a leaving group.
